(19) United States
(12) Patent Application Publication
(10) Pub. No.: US 2003/0166922 A1
(43) Pub. Date: Sep. 4, 2003

(54) Novel cephalosporin compounds and process for preparing the same

(76) Inventors: Yang-Rae Cho, Taejeon (KR); Chang-Seok Lee, Taejeon (KR); Seong-Ho Oh, Taejeon (KR); Eun-Jung Ryu, Taejeon (KR); Kyoung-Sook Paek, Taejeon (KR); Ha-Sik Youn, Taejeon (KR); Yong-Jin Jang, Taejeon (KR); Geun-Tae Kim, Taejeon (KR)

Correspondence Address: BIRCH STEWART KOLASCH & BIRCH, PO BOX 747, FALLS CHURCH, VA, 22040-0747, US

(21) Appl. No.: 10/276,960

(22) PCT Filed: Jun. 14, 2001
(86) PCT No.: PCT/KR01/01020

(30) Foreign Application Priority Data
Jun. 28, 2000 (KR) 2000/35834

(51) Int. Cl. C07D 501/14

(52) U.S. Cl. 540/226

(57) ABSTRACT

The present invention relates to a novel cephalosporin compound in which thiomethylthio chain is introduced into C-3 position of cephem ring and pharmaceutically acceptable non-toxic salt, physiologically hydrolysable ester, hydrate, solvate or isomer thereof. The present invention also relates to a pharmaceutical composition containing the compound and to a process for preparing the compound.

### TECHNICAL FIELD

The present invention relates to a novel cephalosporin compound useful as an antibiotic agent. More specifically, the present invention relates to a novel cephalosporin compound which is useful as an antibacterial agent, and particularly, exhibits a potent activity against strains such as methicillin-resistant Staphylococcus aureus (MRSA), represented by the following formula (I):

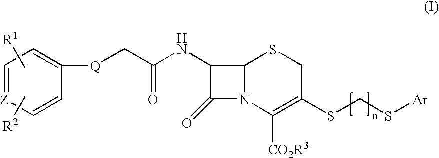

in which

R¹ and R² each independently represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;

R³ represents hydrogen or a carboxy-protecting group;

Q represents S, O, CH₂, NH, or NR wherein R is hydrogen, C₁₋₆ alkyl or benzyl;

Z represents CH or N;

n denotes an integer of 1 or 2; and

Ar represents a heteroaryl group represented by one of the following formulas:

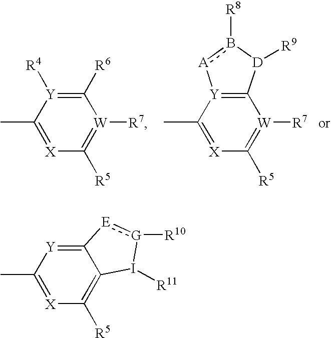

wherein X, Y, W, A, B, D, E, G and I independently of one another represent N or C (or CH), provided that the six-membered ring forms a pyrimidine structure;

R represents hydrogen, C₁₋₄ alkyl, or amino substituted or unsubstituted with a substitutent selected from the group consisting of C₁₋₆ alkyl and C₁₋₆ hydroxyalkyl;

R⁵ and R⁶ each independently represent hydrogen, hydroxy, C₁₋₄ alkyl, C₁₋₆ alkylthio substituted or unsubstituted with a substitutent selected from the group consisting of C₁₋₆ alkyl and C₁₋₆ hydroxyalkyl and C₁₋₆ aminoalkyl, or amino substituted or unsubstituted with a substitutent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen, C₁₋₆ alkyl, or amino substituted or unsubstituted with a substitutent selected from the group consisting of C₁₋₆ alkyl, C₁₋₆ hydroxyalkyl and C₁₋₆ aminoalkyl; and

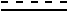

denotes a single bond or a double bond; and pharmaceutically acceptable non-toxic salt, physiologically hydrolysable ester, hydrate, solvate or isomer thereof.

The present invention also relates to a process for preparing the compound of formula (I), as defined above, and to an antibacterial composition containing the compound of formula (I) as an active ingredient.

### BACKGROUND ART

Cephalosporin-based antibiotics have been widely used for the treatment of infectious diseases caused by pathogenic bacteria in human and animals. They are particularly useful for the treatment of diseases caused by bacteria resistant to other antibiotics such as penicillin compounds and for the treatment of penicillin-hypersensitive patients. In most of the cases for treating such infectious diseases, it is preferred to use antibiotics showing an antimicrobial activity against both of gram-positive and gram-negative microorganisms. It has been very well known that such antimicrobial activity of cephalosporin antibiotics is largely influenced by the kind of substituents present at 3- or 7-position of cephem ring. Therefore, through the attempts to develop an antibiotic agent showing a potent antimicrobial activity against broad strains of gram-positive and gram-negative microorganisms, numerous cephalosporin antibiotics having various substituents introduced into 3- or 7-position have been developed up to the present.

For instance, British Patent No. 1,399,086 illustrates broadly and generically cephalosporin derivatives represented by the following formula (II):

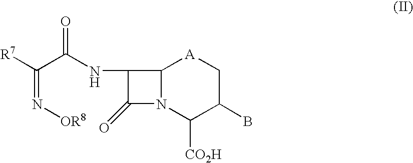

in which

R⁷ represents hydrogen or an organic group;

R⁸is an etherified monovalent organic group, which is linked to oxygen via carbon atom;

A represents —S— or >S→O; and

B represents an organic group.

After development of those compounds, many attempts to develop antibiotic agents having broad antibacterial spectrum have been made and, as a result, numerous cephalosporin antibiotics have been developed. According to the development of them, many studies to introduce acylamido group into 7-position and a certain specific group into C-3 position of the cephem nucleus of formula (II) have also been made in various points of view.

Recently, resistance strains of gram-positive microorganisms, particularly methicillin-resistant Staphylococcus aureus (MRSA) have been recognized as the cause of serious hospital infection and therefore, many attempts have been made to introduce arylthio group into C-3 position to develop cephalosporin compounds showing a potent activity against MRSA.

Thus, Japanese Patent Laid-open No. 98-36375 discloses broadly and generically cephalosporin derivatives represented by the following formula (III) wherein arylthio group is introduced into C-3 position to increase the activity against broad pathogenic strains:

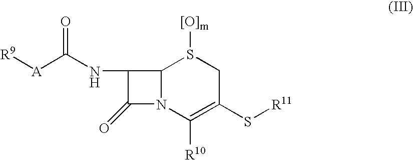

in which

R⁹ represents substituted alkylthio, aryl, arylthio, aryloxy or heterocyclyl group;

A represents protected amino, hydroxy or methylene group;

R¹⁰ represents protected carboxy or carboxylate;

R¹¹ represents halo, cyano, amidino, guanidino, azido, nitro, substituted alkyl, alkenyl, dichloroalkyl, aryl, alkoxy, aryloxy, alkylthio, arylthio, alkylamino, acyl, carbamoyl, carbamoyloxy, alkoxyimino, ureido, alkylsulfinyl, alkylsulfonyl or sulfamoyl, or 2-substituted pyrimidinyl, quinazolinyl, purinyl, pyrazolo[3,4-d]pyrimidinyl, pyrazolo [4,3-d]pyrimidinyl, [1,2,3]triazolo[4,5-d]pyrimidinyl or phtheridinyl; and

m denotes 0 or 1.

In the above patent, various heteroaromatic rings are introduced into thioaryl moiety present at C-3 position; whereas the present invention describes thiomethylthio as a chain present at C-3 position of cephem ring. That is, the above Japanese patent does not mention substituted or unsubstituted pyrimidinyl group or pyridyl group as the substituent introduced into thiomethylthio chain present at C-3 position of cephem ring.

The attempt has been made to develop cephalosporin compounds, which can show a potent activity against serious hospital infection caused by methicillin-resistant Staphylococcus aureus (MRSA), by introducing acyl group into position 7 and pyridine group into C-3 position. Typical example thereof is the compounds of the following formula (IV):

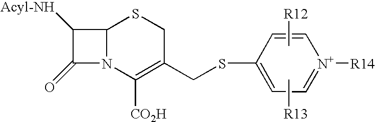

(see, European Patent Laid-open No. EP 96-72742)

in which

Acyl represents Ar-S—CH₂—CO— wherein Ar represents hydrophobic substituted phenyl, pyridyl or benzthiazolyl group;

R¹² and R¹³ each independently represent hydrogen, alkyl or aminoalkylcarbon-ylamino; and

R¹⁴ represents substituted aliphatic, aromatic or arylaliphatic group or a group containing sugar moiety.

In the above European patent, various heteroaromatic rings are introduced into thioaryl moiety present at C-3 position; whereas the present invention describes thiomethylthio as a chain present at C-3 position of cephem ring. That is, the above European patent does not mention substituted or unsubstituted pyrimidinyl group or pyridyl group as the substituent introduced into thiomethylthio chain present at C-3 position of cephem ring.

### DISCLOSURE OF THE INVENTION

Thus, the present inventors have conducted extensive and intensive researches to develop cephalosporin compounds showing broad antibacterial activity against gram-positive microorganisms including MRSA. As a result, we have identified that a certain cephalosporin compound having optionally substituted pyrimidinyl group on thiomethylthio chain at C-3 position meets with the above requirements, and then completed the present invention.

Therefore, the purpose of the present invention is to provide a compound of formula (l), as defined above, and pharmaceutically acceptable non-toxic salt, physiologically hydrolysable ester, hydrate, solvate or isomer thereof.

Further, the purpose of the present invention is to provide a process for preparing the compound of formula (I) and an antibacterial composition containing the compound of formula (I) as an active ingredient.

### DETAILED DESCRIPTION OF THE INVENTION

The present invention relates to a novel cephalosporin compound represented by the following formula (I):

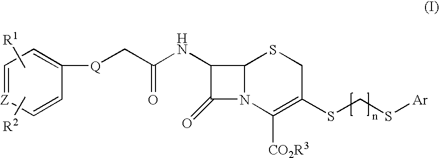

in which

R¹ and R² each independently represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;

R³ represents hydrogen or a carboxy-protecting group;

Q represents S, O, CH₂, NH or NR wherein R is hydrogen, C₁₋₆ alkyl or benzyl;

Z represents CH or N;

n denotes an integer of 1 or 2; and

Ar represents a heteroaryl group represented by one of the following formulas:

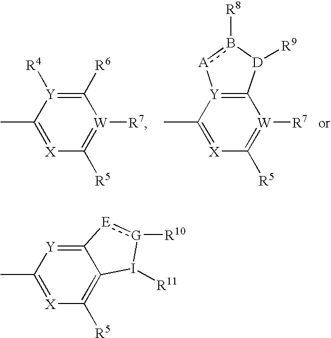

wherein X, Y, W, A, B, D, E, G and I independently of one another represent N or C (or CH), provided that the six-membered ring forms a pyrimidine structure;

R⁴ represents hydrogen, C₁₄ alkyl, or amino substituted or unsubstituted with a substitutent selected from the group consisting of C₁₋₆ alkyl and C₁₋₆ hydroxyalkyl;

R⁵ and R⁶ each independently represent hydrogen, hydroxy, C₁₋₄ alkyl, C₁₋₆ alkylthio substituted or unsubstituted with a substitutent selected from the group consisting of C₁₋₆ alkyl, C₁₋₆ hydroxyalkyl and C₁₋₆ aminoalkyl, or amino substituted or unsubstituted with a substitutent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen, C₁₋₆ alkyl, or amino substituted or unsubstituted with a substitutent selected from the group consisting of C₁₋₆ alkyl, C₁₋₆ hydroxyalkyl and C₁₋₆ aminoalkyl; and

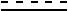

denotes a single bond or a double bond;

and pharmaceutically acceptable non-toxic salt, physiologically hydrolysable ester, hydrate, solvate and isomer thereof.

The compound of formula (I) according to the present invention can be administered in the form of an injectable formulation or an oral formulation depending on the purpose of its use.

Pharmaceutically acceptable non-toxic salts of the compound of formula (I) include salts with inorganic acids such as hydrochloric acid, hydrobromic acid, phosphoric acid, sulfuric acid, etc., salts with organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid, malic acid, etc., or with methanesulfonic acid or para-toluenesulfonic acid, and salts with other acids which have been well-known and widely used in the technical field of penicillins and cephalosporins. These acid addition salts can be prepared according to any of the conventional methods. Further, the compound of formula (I) can also form a non-toxic salt with a base. The base which can be used for this purpose includes inorganic bases such as alkali metal hydroxides (e.g. sodium hydroxide, potassium hydroxide, etc.), alkali metal bicarbonates (e.g. sodium bicarbonate, potassium bicarbonate, etc.), alkali metal carbonates (e.g. sodium carbonate, potassium carbonate, calcium carbonate, etc.), etc., and organic bases such as amino acids.

Examples of physiologically hydrolysable esters of the compound of formula (I) include indanyl, phthalidyl, methoxymethyl, pivaloyloxymethyl, glycyloxymethyl, phenylglycyloxymethyl, 5-methyl-2-oxo-1,3-dioxoren-4-yl methyl esters or other physiologically hydrolysable esters which have been well-known and widely used in the field of penicillins and cephalosporins. These esters can be prepared according to any of the known conventional methods.

Typical examples of the compound of formula (I) according to the present invention include

I-1: (6R,7R)-3 -({[(2,6-diamino-4-pyrimidinyl)sulfanyl]methyl}sulfanyl)-7-({2-[(2, 5-dichlorophenyl)sulfanyl]acetyl}amino)-8-oxo-5-thia-1-azabicyclo[4.2.0]oct-2-ene-2-carboxylic acid,

I-2:

(6R,7R)-3 -({[(2-amino-6-hydroxy-4-pyrimidinyl)sulfanyl]methyl}sulfanyl)-7-({2- [(2,5-dic hlorophenyl)sulfanyl]acetyl}amino)-8-oxo-5-thia-1-azabicyclo[4.2.0]oct-2-ene-2-carboxylic acid,

I-3:

(6R,7R)-3-({[(2-amino-6-hydroxy-4-pyrimidinyl)sulfanyl]methyl}sulfanyl)-7-({2-[(2,6-dic hlorophenyl)sulfanyl]acetyl}amino)-8-oxo-5-thia-1-azabicyclo[4.2.0]oct-2-ene-2-carboxylic acid,

I-4:

(6R,7R)-3-({[(6-amino-2-hydroxy-4-pyrimidinyl)sulfanyl]methyl}sulfanyl)-7-({2-[(2,5-dic hlorophenyl)sulfanyl]acetyl}amino)-8-oxo-5-thia-1-azabicyclo[4.2.0]oct-2-ene-2-carboxylic acid,

I-5: (6R,7R)-3-({[(2,6-diamino-4-pyrimidinyl)sulfanyl]methyl}sulfanyl)-7-{[2-[(2, 5-dichloroanilino)acetyl]amino}-8-oxo-5-thia-1-azabicyclo[4.2.0]oct-2-ene-2-carboxylic acid,

I-6: (6R,7R)-3-({[(2,6-diamino-4-pyrimidinyl)sulfanyl]methyl}sulfanyl)-7-({2-[2, 5-dichloro(methyl)anilino]acetyl}amino)-8-oxo-5-thia-1-azabicyclo[4.2.0]oct-2-ene-2-carboxylic acid,

I-7:

1,4-diamino-2-[({[(6R,7R)-2-carboxy-7-({2-[(2,5-dichlorophenyl)sulfanyl]ac-etyl}amino)-8-oxo-5-thia-1-azabicyclo[4.2.0]oct-2-en-3-yl)sulfanyl}methyl)sulfanyl]pyri-midin-1-ium,

I-8:

(6R,7R)-7-amino-5-[({[2-carboxy-7-({2-[(2,5-dichlorophenyl)sulfanyl]acety-1}amino)-8-o xo-5-thia-1-azabicyclo[4.2.0]oct-2-en-3-yl]sulfanyl}methyl)sulfanyl]-3H-[1,2, 4]triazolo[1,5-c]pyrimidin-4-ium,

I-9:

(6R,7R)-3-({[(4-amino-6,7-dihydro-5H-cyclopenta[d]pyrimidin-2-yl)sulfany-1]methyl}sulf anyl)-7-({2-[(2,5-dichlorophenyl)sulfanyl]acetyl}amino)-8-oxo-5-thia- 1 -aza-bicyclo[4.2.0]oct-2-ene-2-carboxylic acid,

I-10:

(6R,7R)-1,4,6-triamino-2-[({[2-carboxy-7-({2-[(2,5-dichlorophenyl)sulfany-1]acetyl}amin o)-8-oxo-5-thia- 1 -azabicyclo[4.2.0]oct-2-en-3 -yl]sulfanyl}methyl)sulfanyl]p-yrimidin-1-iu m,

I-11:

(6R,7R)-1 ,2-diamino-4-[({(E)-3 -[2-carboxy-7-({2-[(2,5-dichlorophenyl)sulf-anyl]acetyl}a mino)-8-oxo-5-thia-1-azabicyclo[4.2.0]oct-2-en-3-yl]sulfanyl}methyl)sulfan-yl]-6,7-dihyd ro-5H-cyclopenta[d]pyrimidin-1-ium,

I-12:

(6R,7R)-7-({2-[(2,5-dichlorophenyl)sulfanyl]acetyl}amino)-3-[({[2-(ethylsul-fanyl)-6-met hyl-4-pyrimidinyl]sulfanyl}methyl)sulfanyl]-8-oxo-5-thia-1-azabicyclo[4.2. 0]oct-2-ene-2-carboxylic acid,

I-13:

(6R,7R)-3-({[(7-amino-1H-pyrazolo [4,3-d]pyrimidin-5-yl)sulfanyl]methy-1}sulfanyl)-7-({2-[(2,5-dichlorophenyl)sulfanyl]acetyl}amino)-8-oxo-5-thia-1-azabicyc-lo[4.2.0]oct-2-ene-2-carboxylic acid,

I-14:

(6R,7R)-2,7-diamino-5-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-methyl-1H,2H, 3H-[1,2,4]triazolo[1,5-c]pyrimidin-4-ium,

(6R,7R)-2,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-methylpyrimidi n-1-ium,

I-16:

(6R,7R)-3-({[(4,6-diamino-2-pyrimidinyl)sulfanyl]methyl}sulfanyl)-7-({2-[(2,5-dichlorop henyl)sulfanyl]acetyl}amino)-8-oxo-5-thia-1-azabicyclo[4.2.0]oct-2-ene-2-carboxylic acid,

I-17:

(6R,7R)-3-({[(5,6-diamino-4-pyrimidinyl)sulfanyl]methyl}sulfanyl)-7-({2-[(2,5-dichlorop henyl)sulfanyl]acetyl}amino)-8-oxo-5-thia-1-azabicyclo[4.2.0]oct-2-ene-2-carboxylic acid,

I-18:

(6R,7R)-3-({[(4,6-diamino-5-methyl-2-pyrimidinyl)sulfanyl]methyl}sulfan-yl)-7-({2-[(2,5-dichlorophenyl)sulfanyl]acetyl}amino)-8-oxo-5-thia-1-azabicyclo[4.2.0]oc-t-2-ene-2-carb oxylic acid,

I-b 19:

(6R,7R)-1,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-3-methyl-2-(meth ylamino)-1,3,5-triazine-1,3-diium,

I-b 20:

(6R,7R)-2,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-ethyl-1,3,5-tria zine-1,3-diium,

I-21:

(6R,7R)-5-[({[(2-carboxy-7-({2-[(2,5-dichlorophenyl)sulfanyl]acetyl}amino)-8-oxo-5-thia -1-azabicyclo[4.2.0]oct-2-en-3-yl)]sulfanyl}methyl)sulfinyl]-1H-[1,2,4]triazo-lo[3,4-b][1,3 ,5]triazine-4-ium,

I-22:

(6R,7R)-7-({2-[(2,5-dichlorophenyl)sulfanyl]acetyl}amino)-3-[({[6-methyl-2-(methylsulfa nyl)-4-pyrimidinyl]sulfanyl}methyl)sulfanyl]-8-oxo-5-thia-1-azabicyclo[4.2. 0]oct-2-ene-2-carboxylic acid,

I-23:

(6R,7R)-1,4,6-triamino-2-[({[(2-carboxy-7-({2-[(2,5-dichlorophenyl)sulfan-yl]acetyl}amin o)-8-oxo-5-thia-1-azabicyclo[4.2.0]oct-2-en-3-yl)]sulfanyl}methyl)sulfin-yl]-5-methylpyri midin-1-ium,

I-24:

(6R,7R)-3-({[(2,6-diamino-4-pyrimidinyl)sulfanyl]methyl}sulfanyl)-7-({2-[(2,6-dichloro-4-pyridinyl)sulfanyl]acetyl}amino)-8-oxo-5-thia-1-azabicyclo[4.2.0]oct-2-ene-2-carboxylic acid,

I-25:

(6R,7R)-3-({[(4,6-diamino-2-pyrimidinyl)sulfanyl]methyl}sulfanyl)-7-({2-[(2,6-dichloro-4-pyridinyl)sulfanyl]acetyl}amino)-8-oxo-5-thia-1-azabicyclo[4.2.0]oct-2-ene-2-carboxylic acid,

I-26:

(6R,7R)-3-({[(4-amino-1H-pyrazolo[3 ,4-d]pyrimidin-6-yl)sulfanyl]methy-1}sulfanyl)-7-({2-[(2,6-dichloro-4-pyridinyl)sulfanyl]acetyl}amino)-8-oxo-5-thia-1-azabi-cyclo[4.2.0]oct-2-ene-2-carboxylic acid,

I-27:

(6R,7R)-7-({2-[(2,6-dichloro-4-pyridinyl)sulfanyl]acetyl}amino)-8-oxo-3- {[(1H-pyrazolo[3,4-d]pyrimidin-4-yl-sulfanyl)methyl]sulfanyl}-5-thia-1-azabicyclo[4.2. 0]oct-2-ene-2-carboxylic acid,

I-28:

(6R,7R)-3-({[(2-amino-6-hydroxy-4-pyrimidinyl)sulfanyl]methyl}sulfanyl)-7-({2-[(2,6-dic hloro-4-pyridinyl)sulfanyl]acetyl}amino)-8-oxo-5-thia-1-azabicyclo[4.2.0]-oct-2-ene-2-car boxylic acid,

I-29:

(6R,7R)-7-({2-[(2,6-dichloro-4-pyridinyl)sulfanyl]acetyl}amino)-3-[({[2-(ethylsulfanyl)-6-methyl-4-pyrimidinyl]sulfanyl)methyl]sulfanyl}-8-oxo-5-thia-1-azabicy-clo[4.2.0]oct-2-en e-2-carboxylic acid,

I-30:

7-amino-5-[({[(6R,7R)-2-carboxy-7-({2-[(2,6-dichloro-4-pyridinyl)sulfany-1]acetyl}amino )-8-oxo-5-thia-1-azabicyclo[4.2.0]oct-2-en-3-yl]sulfanyl}methyl)sulfanyl]-1H-[1,2,4]triazo lo[1, 5-c]pyrimidin-4-ium,

I-31:

2,7-diamino-5-[({[(6R,7R)-2-carboxy-7-({2-[(2,6-dichloro-4-pyridinyl)sulf-anyl]acetyl}a mino)-8-oxo-5-thia-1-azabicyclo[4.2.0]oct-2-en-3-yl]sulfanyl}methyl)sulfan-yl]-1-methyl-1H-[1,2,4]triazolo[1,5-c]pyrimidin-4-ium,

I-32:

(6R,7R)-7-({2-[(2,6-dichloro-4-pyridinyl)sulfanyl]acetyl}amino)-3- {[({4-hy-droxy-6-[(2-h ydroxyethyl)amino]-2-pyrimidinyl}sulfanyl)methyl]sulfanyl}-8-oxo-5-thia-1-azabicyclo[4. 2.0]oct-2-ene-2-carboxylic acid, and

I-33:

2,4-diamino-6-[({[(6R,7R)-2-carboxy-7-({2-[(2,6-dichloro-4-pyridinyl)sulf-anyl]acetyl}a mino)-8-oxo-5-thia-1-azabicyclo[4.2.0]oct-2-en-3-yl]sulfanyl}methyl)sulfan-yl]-1-methylp yrimidin-1-ium.

According to the present invention, the compound of formula (I):

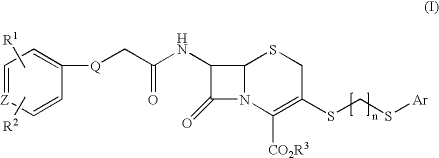

wherein R¹, R², R³, Z, Q, n and Ar are as defined above, or pharmaceutically acceptable non-toxic salt, physiologically hydrolysable ester, hydrate, solvate or isomer thereof can be prepared by a process which comprises

reacting a compound of formula (V):

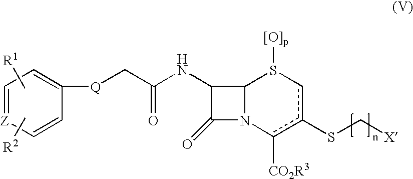

in which R¹, R², R³, Z, Q and n are as defined in the formula (I), p denotes an integer of 0 or 1, and X′ represents halogen atom, with a compound of formula (VI):

HS-Ar (VI)

in which Ar is as defined in the formula (I), optionally following the addition of alkali metal to obtain a compound of formula (VII):

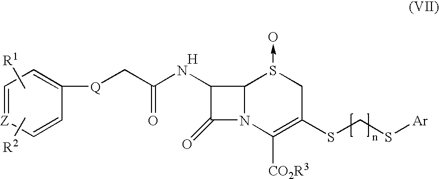

in which R¹, R², R³, Z, Q, n and Ar are as defined in the formula (I),

and reducing S-oxide of the compound of formula (VII). If necessary, the process further comprises the step of removing the acid-protecting group before or after the reaction.

In the process for preparing the compound of formula (I) according to the present invention, the compound of formula (VI) is used in an amount of 1 to 2 moles with respect to one mole of the compound of formula (V).

The reaction temperature can be varied within a broad range and is generally in the range of −10° C. to 50° C., preferably in the range of 20° C. to 35° C.

The process for preparing the compound of formula (I) according to the present invention can be carried out using a solvent. Suitable solvent for this purpose is a non-reactive solvent and includes, for example, dimethylformamide, dimethylsulfoxide, methylene chloride, etc.

In the above process, carboxy-protecting group R³ is desirably the group which can be readily removed under mild condition. Typical examples of carboxy-protecting group R³ include (lower) alkyl ester (e.g. methyl ester, t-butyl ester, etc.), (lower) alkenyl ester (e.g. vinyl ester, allyl ester, etc.), (lower) alkylthio (lower) alkyl ester (e.g. methylthiomethyl ester, etc.), halo (lower) alkyl ester (e.g. 2,2,2-trichloroethyl ester, etc.), substituted or unsubstituted aralkyl ester (e.g. benzyl ester, p-nitrobenzyl ester, p-methoxybenzyl ester, etc.) or silyl ester, etc. These carboxy-protecting groups can be readily removed under mild reaction conditions such as hydrolysis, reduction, etc. to generate a free carboxy group, and appropriately selected depending on the chemical properties of the compound of formula (I).

Leaving group X′ represents, for example, a halogen atom such as chlorine, fluorine and iodine. The halogen atom represented by X′ in formula (V) may be converted into other halogen atoms by the common methods. For example, the compound of formula (V) wherein X′ is iodine atom can be obtained by the reaction of the compound of formula (V) wherein X′ is chlorine atom with alkali metal iodide.

The compound of formula (V) as a starting material can be obtained by reacting a compound of the following formula (VIII):

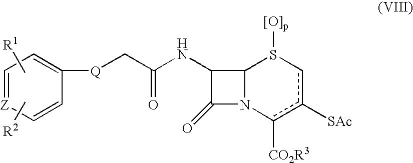

in which R¹, R², R³, Z, Q and p are as defined above, with dihalogenomethane or dihalogenoethane in the presence of a base. Amines such as organic secondary amines and aromatic amines are commonly used as a base, and the dihalogenomethane or dihalogenoethane includes bromochloromethane, bromochloroethane, chloroiodomethane and chloroiodoethane, etc.

The compound of formula (VIII) as an intermediate of the present invention can be prepared by activating a compound of the following formula (IX):

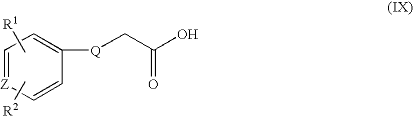

in which R¹, R², Q and Z are as defined above, and salt thereof with an acylating agent, and then reacting the resulting activated compound with a compound of the following formula (X):

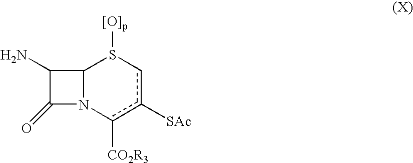

in which R³ and p are as defined above.

The dotted lines in the formulas (V), (VIII) and (X) represent each of 2-cephem and 3-cephem compounds, or their mixture. That is, the dotted line in the formula (X) means that the compound of formula (X) can exist as compounds of the following formulas (Xa) and (Xb):

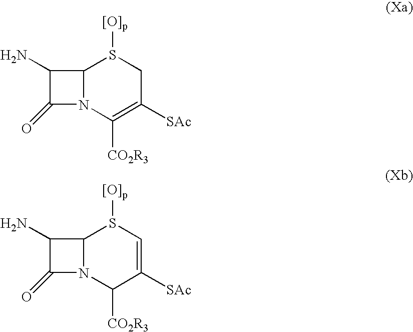

wherein R³ and p are as defined above, respectively, or mixture thereof.

In preparing the compound of formula (VIII), an acylated derivative as the activated form of the compound of formula (IX) includes acid chlorides, acid anhydrides, mixed acid anhydrides (preferably, acid anhydrides formed with methylchloroformate, mesitylenesulfonyl chloride, p-toluenesulfonyl chloride or chlorophosphate) or activated esters (preferably, esters formed from the reaction with N-hydroxybenzotriazole in the presence of a condensing agent such as dicyclohexylcarbodiimide), etc. In addition, the acylation reaction can also be practiced by using a free acid compound of formula (IX) in the presence of a condensing agent such as dicyclohexylcarbodidimide or carbonyldiimidazole. Further, the acylation reaction is well practiced generally in the presence of a tertiary amine, preferably an organic base such as triethylamine, dimethylaniline, pyridine, etc., or an inorganic base such as sodium bicarbonate, sodium carbonate, etc. The solvent which can be used in this reaction includes halogenated hydrocarbon such as methylene chloride, chloroform, etc., tetrahydrofuran, acetonitrile, dimethylformamide or dimethyl acetamide. The mixed solvent comprising two or more solvents selected from the above can also be used. The reaction can also be carried out in an aqueous solution.

The reaction temperature in the acylation reaction is in the range of −50° C. to 50° C., preferably in the range of −30° C. to 20° C. The acylating agent for the compound of formula (IX) can be used in an equimolar amount or a slightly excessive amount, i.e. in an amount of 1.05 to 1.2 equivalents, with respect to an equivalent of the compound of formula (X).

In preparing the compound of formula (I) as defined above, the amino-protecting group or the acid-protecting group present in the compound of formula (V) can be removed by any of the conventional methods widely known in the field of cephalosporins. That is, the protecting groups can be removed by hydrolysis or reduction. Acid hydrolysis is useful for removing tri(di)phenylmethyl group or alkoxycarbonyl group and is carried out using an organic acid such as formic acid, trifluoroacetic acid, p-toluenesulfonic acid, etc., or an inorganic acid such as hydroxhloric acid, etc.

The resulting product from the above processes can be treated with various methods such as recrystallization, electrophoresis, silica gel column chromatography or ion exchange resin chromatography to separate and purify the desired compound of formula (I).

The present invention also relates to a pharmaceutical composition containing the compound of formula (I) or pharmaceutically acceptable salt thereof as an active ingredient, together with a pharmaceutically acceptable carrier.

The compound according to the present invention can be administered in the form of an injectable formulation or an oral formulation depending on the purpose of its use.

The compound of formula (I) of the present invention can be formulated using known pharmaceutically acceptable carriers and excipients according to the known method to prepare a unit dosage form or introduce into a multi-dosage container. The formulations can be in the form of a solution, suspension or emulsion in an oil or aqueous medium and can contain conventional dispersing agent, suspending agent or stabilizing agent. In addition, the formulation can also be in the form of a ready-to-use dry powder which can be used by dissolving with a steril, pyrogen-free water before its use. The compound of formula (I) can also be formulated in the form of a suppository by using conventional suppository bases such as cocoa butter or other glycerides. Solid dosage form for oral administration includes capsules, tablets, pills, powders and granules, with capsules and tablets being particularly useful. For the tablets and pills, it is preferred to provide an enteric coating. Solid dosage form can be prepared by mixing the active compound of formula (T) according to the present invention with one or more inert diluents such as sucrose, lactose, starch, etc., and carriers including lubricants such as magnesium stearate, disintegrating agents, binders, etc.

If necessary, the compound of the present invention can be administered in combination with other antibacterial agent such as penicillins or other cephalosporins.

In formulating the compound of formula (I) according to the present invention into the unit dosage form, it is preferred that the unit dosage form contains the compound of formula (I) as an active ingredient in an amount of about 50 to 1,500 mg. The dosage of the compound of formula (I) is suitably selected under the physician's prescription depending on various factors including weight and age of patient, particular conditions and severity of diseases to be treated, etc. However, the daily dosage for the treatment of adult man generally corresponds to about 500 to 5,000 mg of the compound of formula (I) depending on the frequency and intensity of administration. For intramuscular or intravenous injection to adult man, a total daily dosage in the range of about 150 to 3,000 mg is generally sufficient. However, in case of infections caused by some pathogenic strains, it may be preferred to more increase the daily doage.

The compound of formula (I) and its non-toxic salt (preferably salts with alkali metals, alkali earth metals, inorganic acids, organic acids and amino acids) according to the present invention exhibit a potent antimicrobial activity and a broad antibacterial spectrum against broad pathogenic microorganisms including various gram-positive strains and therefore, are very useful for the prevention and treatment of diseases caused by bacterial infection in animals including human being.

The present invention will be more specifically illustrated by the following preparations and examples. However, it should be understood that these preparations and examples are provided only to help the clear understanding of the present invention but do not intend to limit the present invention in any manner.

### BEST MODE FOR CARRYING OUT THE INVENTION

Preparation 1: Synthesis of benzhydryl (6R,7R)-3-(acetylsulfanyl)-7-({2-[(2,5-dichlorophenyl)sulfanyl]acetyl}amino)-8-oxo-5-thia-1-azabicyclo[4.2.0]oct-2-ene-2-carboxylate

1.5 g(3.08 mmol) of benzhydryl (6R,7R)-3-(acetylsulfanyl)-7-amino-8-oxo-5-thia-1-azabicyclo[4.2.0]oct-2-ene-2-carboxyl ate hydrochloric acid salt and 0.73 g(3.08 mmol) of 2,5-dichlorophenylthioacetic acid were dissolved in 20 ml of dichloromethane. After the temperature of the reaction vessel was lowered to −30° C., 0.55 ml(7.70 mmol) of pyridine and 0.32 ml(3.39 mmol) of phosphoryloxy chloride were slowly added dropwise to the reaction mixture. The reaction vessel was gradually warmed to 0° C. with stirring for 3.5 hours. The mixture was diluted with an excess of ethylacetate, washed with saturated ammonium chloride solution, 5% sodium bicarbonate solution and brine one by one, dried over anhydrous magnesium sulfate and filtered. After the filtrate was evaporated under reduced pressure, the resulting residue was purified by column chromatography to give 0.9 g(yield: 43.6%) of the title compound.

¹H NMR(CDCl₃) δ 7.35-7.25(12H, m), 7.19(1H, m), 7.01(1H, s), 5.83˜5.82(1H, m), 5.07˜5.06(1H, m), 3.78˜3.74(3H, Abq, m), 3.35˜3.31(1H, Abq, J=18.3 Hz), 2.09(3H, s)

Mass(m/e) 658

Preparation 2: Synthesis of benzhydryl (6R,7R)-3-{(chloromethyl)sulfanyl}-7-({2-[(2,5-dichlorophenyl)sulfanyl]acetyl}amino) -8-oxo-5-thia-1-azabicyclo[4.2.0]oct-2-ene-2-carboxylate

0.9 g(1.343 mmol) of benzhydryl (6R,7R)-3-(acetylsulfanyl)-7-({2-[(2,5-dichloro-phenyl)sulfanyl]acetyl}amino)-8-oxo-5-th ia-1-azabicyclo[4.2.0]oct-2-ene-2-carboxylate was dissolved in 10 ml of dimethylformamide, and then the reaction vessel was cooled to −20° C. Subsequently, 0.07 ml(0.805 mmol) of morpholine was slowly added dropwise to the reaction mixture. After stirring at −20° C. for 1 hour, the mixture was diluted with ethylacetate, washed with 1% hydrochloric acid solution and brine, dried over anhydrous magnesium sulfate, and filtered. The resulting filtrate was evaporated under reduced pressure. The remaining residue was dissolved in 10 ml of dimethylformamide and the temperature of the reaction vessel was lowered to −20° C. Subsequently, to the resulting solution were slowly added 0.23 ml(3.08 mmol) of chloroiodomethane and 0.16 ml(0.938 mmol) of diisopropylethylamine. After stirring at −20° C. for 24 hours, the mixture was diluted with ethylacetate, washed with 1% hydrochloric acid solution and brine, dried over anhydrous magnesium sulfate, and concentrated. After the filtrate was evaporated under reduced pressure, the resulting residue was purified by column chromatography to give 0.6 g(yield: 66.0%) of the title compound.

¹H NMR(CDCl₃) δ 7.45˜7.42(1H, d), 7.34˜7.31(11H, m), 7.14˜7.12(1H, d), 6.93(1H, s), 5.74˜5.71(1H, dd, J=5.0 Hz), 5.01˜5.00(1H, d, J=5.05 Hz), 4.71˜4.60(2H, q, J=12.8 Hz), 3.81˜3.65(4H, m)

Mass(m/e) 664

### EXAMPLE 1

Synthesis of (6R,7R)-3-({[(2,6-diamino-4-pyrimidinyl)sulfanyl]methyl}su-lfanyl)-7-({2-[(2,5-dichlo rophenyl)sulfanyl]acetyl}amino)-8-oxo-5-thia-1-azabicyclo[4. 2.0]oct-2-ene-2-carboxylic acid

After 0.41 g(0.609 mmol) of benzhydryl (6R,7R)-3- {(chloromethyl)sulfanyl}-7-({2-[(2,5-dichlorophenyl)sulfanyl]acetyl}amino)-8-oxo-5-thia-1-azabicyclo[4.2.0]oct-2-ene-2-carboxylate was dissolved in 5 ml of dimethylformamide, 0.18 g(1.218 mmol) of sodium iodide was added thereto and subsequently 0.175 g(0.7308 mmol) of 2,4-diamino-6-mercaptopyrimidine ½ sulfuric acid salt was added to the resulting mixture. The mixture was stirred at room temperature for 24 hours. The mixture was diluted with an excess of ethylacetate, washed with brine three times, dried over anhydrous magnesium sulfate, filtered and concentrated. After the filtrate was evaporated under reduced pressure, the resulting residue was purified with diethyl ether and dried under nitrogen atmosphere to obtain 0.46 g of a solid. The obtained solid was deprotected with trifluoroacetic acid and anisole, and then purified by high pressure fractional liquid chromatography to give 0.1 g(yield through two steps: 27.7%) of the title compound.

¹H NMR(DMSO-d₆) δ 9.31˜9.30(1H, d), 7.55˜7.47(2H, m), 7.26(1H, d), 5.95(1H, s), 5.66˜5.65(1H, dd, J=4.55 Hz), 5.14˜5.13(1H, d, J=4.55 Hz), 4.69˜4.64(2H, q), 3.98(2H, s), 3.86˜3.85(2H, d)

Mass(m/e) 604

### EXAMPLE 2

Synthesis of (6R,7R)-3-({[(2-amino-6-hydroxy-4-pyrimidinyl)sulfan-yl]methyl}sulfanyl)-7-({2-[(2,5-dichlorophenyl)sulfanyl]acetyl}amino)-8-oxo-5-thia-1-azabicyclo [4.2.0]oct-2-ene-2-ca rboxylic acid

The title compound(yield through two steps: 18.5%) was obtained according to the same procedure as Example 1.

¹H NMR(DMSO-d₆) δ 3.40(1H, d, 16.8 Hz), 3.72(1H, d, 17 Hz), 3.95(2H, s), 4.40(2H, m), 4.90(1H, d, 4.4 Hz), 5.50(1H, m), 7.20(1H, m), 7.45(2H, m), 9.35(1H, d, 8.0 Hz)

Mass(m/e) 605

### EXAMPLE 3

Synthesis of (6R,7R)-3-({[(2-amino-6-hydroxy-4-pyrimidinyl)sulfan-yl]methyl}sulfanyl)-7-({2-[(2,6 -dichlorophenyl)sulfanyl]acetyl}amino)-8-oxo-5-thia-1-azabicyclo[4.2.0]oct-2-ene-2-ca rboxylic acid

The title compound(yield through two steps: 22.0%) was obtained according to the same procedure as Example 1.

¹H NMR(DMSO-d₆) δ 3.65(1H, d, 16.5 Hz), 3.75(1H, d, 16.8 Hz), 3.85(2H, s), 4.50(2H, m), 5.15(1H, d, 4.6 Hz), 5.50(1H, s), 5.55(1H, m), 7.48(1H, m), 7.58(2H, m), 9.18(1H, d, 8.2 Hz)

Mass(m/e) 605

### EXAMPLE 4

Synthesis of (6R,7R)-3-({1(6-a mino-2-hydroxy-4-pyrimidinyl)sulfan-yl]methyl}sulfanyl)-7-({2-[(2,5-dichlorophenyl)sulfanyl]acetyl}amino)-8-oxo-5-thia-1-azabicyclo[4.2.0]oct-2-ene-2-ca rboxylic acid

The title compound(yield through two steps: 15.5%) was obtained according to the same procedure as Example 1.

¹H NMR(DMSO-d₆) δ 3.42(1H, d, 16.5 Hz), 3.75(1H, d, 16.6 Hz), 3.90(2H, s), 4.52(2H, m), 5.00(1H, d, 4.8 Hz), 5.62(1H, m), 7.25(1H, m), 7.55(2H, m), 9.20(1H, d, 8.5 Hz)

Mass(m/e) 605

### PREPARATION 3

Synthesis of benzhydryl (6R,7R)-3-(acetylsulfanyl)-7-{12-(2,5-dichloroanilino)acetyl]amino}-8-oxo-5-thia-1-aza bicyclo[4.2.0]oct-2-ene-2-carboxylate

The title compound(yield through two steps: 73.5%) was obtained according to the same procedure as Preparation 1.

¹H NMR(CDCl₃) δ 2.05(3H, s), 3.33(1H, d, 17.9 Hz), 3.79(1H, d, 18 Hz), 3.86(1H, dd, 4.6 Hz, 17.9 Hz), 3.99(1H, dd, 6.9 Hz, 17.8 Hz), 4.95(1H, m), 5.11(1H, d, 5 Hz), 5.90(1H, m), 6.50(1H, d, 2.3 Hz), 6.74(1H, dd, 2.3 Hz, 8.3 Hz), 6.95(1H., s), 7.30(11H, m)

Mass(m/e) 641

### PREPARATION 4

Synthesis of 2-[2,5-dichloro(methyl)anilino]acetic acid

1 g of 2-(2,5-dichloroanilino)acetic acid, 1.9 g of potassium carbonate and 1.43 ml of methyl iodide were dissolved in 10 ml of DMF. The mixture was stirred at about 80° C. overnight. After removal of the solvent under reduced pressure, the resulting residue was purified by column chromatography. The obtained compound was dissolved in methanol, and then 3 ml of 1N aqueous sodium hydroxide solution was added thereto. The mixture was stirred for 3 hours. After removal of the solvent under reduced pressure. The resulting residue was dissolved in water and acidified using hydrochloric acid to obtain a solid. The obtained solid was dried to give the title compound(yield: 70%).

¹H NMR(CDCl₃) δ 2.95(3H, s), 3.92(2H, s), 6.99(1H, dd, 2.3 Hz, 8.25 Hz), 7.14(1H, d, 2.3 Hz), 7.26(1H, d, 8.3 Hz)

Mass(m/e) 233

### EXAMPLE 5

Synthesis of (6R,7R)-3-({[(2,6-diamino-4-pyrimidinyl)sulfanyl]meth-yl}sulfanyl)-7-{12-[(2,5-dichlo roanilino)acetyl]amino}-8-oxo-5-thia-1-azabicyclo [4.2.0]oct-2-ene-2-carboxylic acid

The title compound(yield through two steps: 26.0%) was obtained according to the same procedure as Example 1.

¹H NMR(D₂O) δ 3.44(1H, d, 16.9 Hz), 3.68(1H, d, 17 Hz), 4.02(2H, q, 29.8 Hz), 4.25(2H, m), 5.22(1H, d, 4.6 Hz), 5.52(1H, d, 4.4 Hz), 5.89(1H, s), 6.55(1H, m), 6.66(2H., m), 7.18(1H, m)

Mass(m/e) 587

### PREPARATION 5

Synthesis of benzhydryl (6R,7R)-3-(acetylsulfanyl)-7-({2-[2,5-dichlo-ro(methyl)anilino]acetyl}amino}-8-oxo-5-thia-1-azabicyclo [4.2.0]oct-2-ene-2-carbox-ylate

The title compound(yield through two steps: 70.5%) was obtained according to the same procedure as Preparation 1.

¹H NMR(CDCl₃) δ 2.08(3H, s), 2.82(3H, s), 3.40(1H, d, 17.3 Hz), 3.60(1H, d, 17.4 Hz), 3.70(1H, d, 18 Hz), 3.85(1H, d, 17.3 Hz), 5.18(1H, d, 5.1 Hz), 6.00(1H, m), 6.97(1H, s), 7.10(1H, d, 2.3 Hz), 7.30(12H, m), 7.97(1H, d, 9.7 Hz)

Mass(m/e) 655

### PREPARATION 6

Synthesis of 2-(2,5-dichloroanilino)acetic acid

10 g of 2,5-dichloroaniline and 6.2 g of glyoxylic acid were dissolved in 100 ml of methanol. The mixture was cooled to 0° C. and stirred for 40 minutes. 4.5 g of sodium cyanoborohydride was slowly added dropwise and the resulting mixture was stirred at room temperature for 3 hours. After removal of the solvent reduced pressure, to the resulting residue was added an excess of diethyl ether. The organic layer was washed with dilute hydrochloric acid and water, dried over anhydrous magnesium sulfate, and filtered. After the resulting filtrate was evaporated under reduced pressure, the remaining residue was solidified using hexane to give the title compound(yield: 60%).

¹H NMR(CDCl₃) δ 3.92(2H, d, 5.5 Hz), 5.86(1H, m), 6.66(2H, m), 7.26(1H, m) Mass(m/e) 219

### EXAMPLE 6

Synthesis of (6R,7R)-3-({[(2,6-diamino-4-pyrimidinyl)sulfanyl]meth-yl}sulfanyl)-7-({2-[2,5-dichlor o(methyl)anilino]acetyl}amino)-8-oxo-5-thia-1-azabicyc-lo[4.2.0]oct-2-ene-2-carboxyli c acid

The title compound(yield through two steps: 21.5%) was obtained according to the same procedure as Example 1.

¹H NMR(D₂O) δ 2.80(3H, s), 3.57(1H, d, 17.4 Hz), 3.80(1H, d, 17 Hz), 3.82(2H, s), 4.38(2H, q, 14.7 Hz), 5.12(1H, d, 4.5 Hz), 5.67(1H, d, 4.7 Hz), 6.01(1H, s), 7.05(1H, m), 7.25(1H, s), 7.35(1H, m)

Mass(m/e) 601

### EXAMPLE 7

Synthesis of 1,4-diamino-2-[({[(6R,7R)-2-carboxy-7-({2-[(2,5-dichloroph-enyl)sulfanyl]acetyl}amin o)-8-oxo-5-thia-1-azabicyclo[4.2.0]oct-2-en-3-yl)sulfanyl}m-ethyl)sulfanyl]-pyrimidin-1-ium

The title compound(yield through two steps: 14.0%) was obtained according to the same procedure as Example 1.

¹H NMR(DMSO-d₆) δ 3.42(1H, d, 17 Hz), 3.70(1H, d, 17 Hz), 3.94(2H, s), 4.42(2H, s), 4.88(1H, d, 4.6 Hz), 5.48(1H, m), 6.50(1H, d, 7.3 Hz), 6.70(1H, d, 7.3 Hz), 7.26(1H, s), 7.49(3H, m), 8.01(1H., d, 7.3 Hz), 9.20(1H, d, 8.3 Hz)

Mass(m/e) 605

### EXAMPLE 8

Synthesis of (6R,7R)-7-amino-5-[({[2-carboxy-7-({2-[(2,5-dichlorophen-yl)sulfanyl]acetyl}amino)-8-oxo-5-thia-1-azabicyclo [4.2.0]oct-2-en-3-yl]sulfanyl}meth-yl)sulfanyl]-3H-[1,2,4]triaz olo[1,5-c]pyrimidin-4-ium

The title compound(yield through two steps: 13.2%) was obtained according to the same procedure as Example 1.

¹H NMR(DMSO) δ 3.47˜3.50(1H, ABq, 16.9 Hz), 3.70˜3.73(1H, ABq, 16.9 Hz), 3.91(s, 2H), 4.64˜4.69(2H, q, 12.8 Hz), 4.93˜4.94(1H, d, 5.0 Hz), 5.67˜5.48(1H, m), 6.16(1H, s), 6.85(1H, s), 7.23˜7.24(1H, d, 7.35 Hz), 7.45˜7.49(2H, m), 8.15(1H, s), 9.20˜9.22(1H, d, 8.25 Hz)

Mass(m/e) 630

### EXAMPLE 9

Synthesis of (6R,7R)-3-({[(4-amino-6,7-dihydro-5H-cyclopenta[d]pyrim-idin-2-yl)sulfanyl]methyl}sulfanyl)-7-({2-[(2,5-dichlorophenyl)sulfanyl]acetyl}amino)-8-oxo-5-thia-1-azabicyclo [4.2.0]oct-2-ene-2-carboxylic acid

The title compound(yield through two steps: 6.7%) was obtained according to the same procedure as Example 1.

¹H NMR(DMSO) δ 1.70(4H, br, s), 2.25(2H, br, s), 3.54˜3.58(1H, ABq, 16.95 Hz), 3.68˜3.71(1H, ABq, 16.05 Hz), 3.89˜4.02(2H, q, 16.95 Hz), 4.47(2H, br, s), 4.93˜4.94(1H, d, 4.15 Hz), 5.47(1H, m), 6.68(1H, br, s), 7.23˜7.25(1H, d, 7.3 Hz), 7.46˜7.50(2H, m), 9.20˜9.22(1H, d, 7.8 Hz)

Mass(m/e) 629

### EXAMPLE 10

Synthesis of (6R,7R)-1,4,6-triamino-2-[({[2-carboxy-7-({2-[(2,5-dichlo-rophenyl)sulfanyl]acetyl}a mino)-8-oxo-5-thia-1-azabicyclo [4.2.0]oct-2-en-3-yl]sulfan-yl}methyl)sulfanyl]pyrimid in-1-ium

The title compound(yield through two steps: 20.0%) was obtained according to the same procedure as Example 1.

¹H NMR(DMSO) δ 3.68˜3.72(1H, ABq, 17.9 Hz), 3.91(2H, br, s), 4.38(2H, br, m), 4.86(1H, m), 5.47˜5.51(1H, m), 6.04(1H, s), 7.25(1H, d, 7.3 Hz), 7.49(2H, m), 8.09(2H, br, m), 9.22(1H, d, 7.85 Hz)

Mass(m/e) 620

### EXAMPLE 11

(6R,7R)-1,2-diamino-4-[({(E)-3-[2-carboxy-7-({2-[(2,5-dichlorophenyl)s-ulfanyl]acetyl }amino)-8-oxo-5-thia-1-azabicyclo [4.2.0]oct-2-en-3-yl]sulfanyl}methyl)s-ulfanyl]-6,7-d ihydro-5H-cyclopenta[d]pyrimidin-1-ium

The title compound(yield through two steps: 5.2%) was obtained according to the same procedure as Example 1.

¹H NMR(DMSO) δ 2.11˜2.12(2H, m), 2.75(2H, m), 2.87(1H, m), 3.05(1H, m), 3.55˜3.60(1H, ABq, 16.95 Hz), 3.60˜3.68(1H, ABq, 16.95 Hz), 3.91(2H,s), 4.43˜4.45(2H, m), 4.88˜4.89(1H,d, 4.58 Hz), 5.48˜5.49(1H, m), 6.52(1H, s), 7.24˜7.25(1H, d, 7.8 Hz), 7.46˜7.49(2H, m), 8.47(1H, s, br), 9.21˜923(1H, d, 7.8 Hz)

Mass(m/e) 645

### EXAMPLE 12

Synthesis of (6R,7R)-7-{(2-[(2,5-dichlorophenyl)sulfanyl]acetyl}amino)-3-[({[2-(ethylsulfanyl)-6-m ethyl-4-pyrimidinyl]sulfanyl}methyl)sulfanyl]-8-oxo-5-thia-1-azabicyclo [4.2.0]oct-2-e ne-2-carboxylic acid

The title compound(yield through two steps: 17.0%) was obtained according to the same procedure as Example 1.

¹H NMR(DMSO) δ 1.29˜1.32(3H, t), 2.32(3H, s), 3.10(2H, q), 3.46˜3.49(1H, ABq, 16.04 Hz), 3.67˜3.71(1H, ABq, 16.04 Hz), 3.91(2H, s), 4.53(1H, d, 12.83 Hz), 4.61(1H, d, 12.83 Hz), 4.94˜4.95(1H, d, 4.58 Hz), 5.48˜5.49(1H, m), 7.15(1H, s), 7.23˜7.26(1H, d, 8.2 Hz), 7.46˜7.49(2H, m), 9.20˜9.22(1H, d, 7.75 Hz)

Mass(m/e) 648

### EXAMPLE 13

Synthesis of (6R,7R)-3-({1(7-amino-1H-pyrazolo [4,3-d pyrimidin-5-yl)sulfanyl]methyl}sulfanyl)-7-({2-[(2,5-dichlorophenyl)sulfanyl]acetyl}amino)-8-oxo-5-thia-1-azabicyclo [4.2.0]oct-2-ene-2-carboxylic acid

The title compound(yield through two steps: 17.0%) was obtained according to the same procedure as Example 1.

¹H NMR(DMSO) δ 3.49˜3.53(1H, ABq, 16.96 Hz), 3.69˜3.72(1H, ABq, 16.50 Hz), 3.91(2H, s), 4.44˜4.50(2H, q, 12.83 Hz), 4.93˜4.94(1H, d, 4.58 Hz), 4.68(1H, m), 7.25(1H, d, 8.2 Hz), 7.45˜7.47(1H, d, 8.7 Hz), 7.5(1H, s), 7.99(1H, s), 9.19˜9.20(1H, d, 8.25 Hz)

Mass(m/e) 629

### EXAMPLE 14

Synthesis of (6R,7R)-2,7-diamino-5-[({[2-carboxy-7-({2-[(2,5-dichlor-ophenyl)sulfanyl]acetyl}amin o)-8-oxo-5-thia-1-azabicyclo [4.2.0]oct-2-en-3-yl]sulfany-1}methyl)sulfanyl]-1-methyl-1H,2H,3H-[1,2,4]triazolo[1,5-cl pyrimidin-4-ium

The title compound(yield through two steps: 2.8%) was obtained according to the same procedure as Example 1.

¹H NMR(DMSO) δ 3.79(1H, d, 17 Hz), 3.92(2H, s), 4.69˜4.75(2H, q, 13.75 Hz), 5.0(1H, d, 4.8 Hz), 5.58˜4.49(1H, m), 6.22(1H, s), 7.25(1H, d, 8.3 Hz), 7.46˜7.48(2H, m), 7.75(2H, s, br), 7.96(2H, br, m), 9.28˜9.30(1H, d, 8.25 Hz)

Mass(m/e) 661

### EXAMPLE 15

Synthesis of (6R,7R)-2,4-diamino-6-[({[2-carboxy-7-({2-[(2,5-dichloroph-enyl)sulfanyl]acetyl}amin o)-8-oxo-5-thia-1-azabicyclo [4.2.0]oct-2-en-3-yl]sulfanyl}m-ethyl)sulfanyl]-1-methylp yrimidin-1-ium

The title compound(yield through two steps: 1.1%) was obtained according to the same procedure as Example 1.

¹H NMR(DMSO) δ 3.41˜3.43(1H, ABq, 16.95 Hz), 3.72˜3.75(1H, ABq, 16.5 Hz), 3.92(2H, s), 4.44˜4.47(2H, m), 5.01˜5.02(1H,d, 4.55 Hz), 5.53˜5.56(1H, m), 7.25(1H, d, 8.25 Hz), 7.46˜7.49(2H, m), 8.18(1H, br, m), 9.24˜9.25(1H, d, 8.20 Hz)

Mass(m/e) 620

### EXAMPLE 16

Synthesis of (6R,7R)-3-({i(4,6-diamino-2-pyrimidinyl)sulfanyl]meth-yl}sulfanyl)-7-({2-[(2,5-dichlo rophenyl)sulfanyl]acetyl}amino)-8-oxo-5-thia-1-azabicy-clo[4.2.0]oct-2-ene-2-carboxy lic acid

The title compound(yield through two steps: 7.4%) was obtained according to the same procedure as Example 1.

¹H NMR(D₂O) δ 7.46˜7.26(2H, m), 7.20˜7.14(1H, m), 5.52(1H, d, 4.6 Hz), 5.49(1H, s), 5.00(1H, d, 4.6 Hz), 4.47(2H, dd), 3.96˜3.88(2H, m), 3.74(1H, d, 17.3 Hz), 3.48(1H, d, 17.3 Hz)

Mass(m/e) 604

### EXAMPLE 17

Synthesis of (6R,7R)-3-({[(5,6-diamino-4-pyrimidinyl)sulfanyl]meth-yl}sulfanyl)-7-({2-[(2,5-dichlo rophenyl)sulfanyl]acetyl}amino)-8-oxo-5-thia-1-azabicy-clo[4.2.0]oct-2-ene-2-carboxy lic acid

The title compound(yield through two steps: 3.9%) was obtained according to the same procedure as Example 1.

¹H-NMR(DMSO-d₆) δ 7.50˜7.43(2H, m), 7.26˜7.23(1H, m), 5.46˜5.43(1H, m), 4.85(1H, d, 4.3 Hz), 4.60˜4.41(2H, m), 4.56(1H, s), 3.91˜3.86(2H, m), 3.63˜3.38(2H, m)

Mass(m/e) 604

### EXAMPLE 18

Synthesis of (6R,7R)-3-({[(4,6-diamino-5-methyl-2-pyrimidinyl)sulfan-yl]methyl}sulfanyl)-7-({2-1(2,5-dichlorophenyl)sulfanyl]acetyl}amino)-8-oxo-5-thia-1-azabicyclo [4.2.0]oct-2-ene-2-carboxylic acid

The title compound(yield through two steps: 5.6%) was obtained according to the same procedure as Example 1.

¹H-NMR(D₂O) δ 7.34˜7.13(2H, m), 7.00˜6.97(1H, m), 5.38(1H, d, 4.4 Hz), 4.84(1H, d, 4.8 Hz), 4.40˜4.17(2H, m), 3.61˜3.24(4H, m), 1.80(3H, s)

Mass(m/e) 618

### EXAMPLE 19

Synthesis of (6R,7R)-1,4-diamino-6-[({[2-carboxy-7-({2-[(2,5-dichloroph-enyl)sulfanyl]acetyl}amin o)-8-oxo-5-thia-1-azabicyclo[4.2.0]oct-2-en-3-yl]sulfanyl}m-ethyl)sulfanyl]-3-methyl-2-(methylamino)-1,3,5-triazine-1,3-diium

The title compound(yield through two steps: 5.5%) was obtained according to the same procedure as Example 1.

¹H NMR(DMSO d₆) δ 9.22(1H, d, 6.8 Hz), 7.69(1H, s), 7.49˜7.46(2H, m), 7.28˜7.23(1H, m), 5.50˜5.42(1H, m), 4.82˜4.76(1H, m), 4.48˜4.41(2H, m), 3.91˜3.89(2H, m), 3.71˜3.65(2H, m), 3.12(3H, d, 5.9 Hz), 2.05(3H, s)

Mass(m/e) 650

### EXAMPLE 20

Synthsis of (6R,7R)-2,4-diamino-6-[({[2-carboxy-7-({2-[(2,5-dichloroph-enyl)sulfanyl]acetyl}amin o)-8-oxo-5-thia-1-azabicyclo [4.2.0]oct-2-en-3-yl]sulfanyl}m-ethyl)sulfanyl]-1-ethyl-1,3, 5-triazine-1,3-diium

The title compound(yield through two steps: 2.5%) was obtained according to the same procedure as Example 1.

¹H NMR(DMSO d₆) δ 9.23(1H, d, 7.8 Hz), 8.2˜7.7(2H, m), 7.49˜7.23(3H, m), 5.62(1H, s), 5.56˜5.50(1H, m), 4.92(1H, d, 5.0 Hz), 4.60(2H, q, 6.8 Hz), 4.10˜3.80(2H, m), 3.65˜3.55(2H, m), 1.21(3H, t, 6.8 Hz)

Mass(m/e) 635

### EXAMPLE 21

Synthsis of (6R,7R)-5-[({[(2-carboxy-7-({2-[(2,5-dichlorophenyl)sulfan-yl]acetyl}amino)-8-oxo-5-t hia-1-azabicyclo [4.2.0]oct-2-en-3-yl)]sulfanyl}methyl)sulf-inyl]-1H-[1,2,4]triazolo[3,4-b][1,3,5]triazine-4-ium

The title compound(yield through two steps: 1.9%) was obtained according to the same procedure as Example 1.

¹H NMR(DMSO d₆) δ 9.31˜9.23(1H, d, 8.2 Hz), 8.75(1H, s), 8.27(1H, s), 7.49˜7.45(2H, m), 7.24˜7.22(1H, m), 5.49˜5.40(1H, m), 4.99˜4.96(1H, m), 4.84(1H, d, 13.3 Hz), 4.75(1H, d, 13.3 Hz), 3.98˜3.90(2H, m), 3.72(1H, d, 16.9 Hz), 3.56(1H, d, 16.9 Hz)

Mass(m/e) 616

### EXAMPLE 22

Synthsis of (6R,7R)-7-({2-[(2,5-dichlorophenyl)sulfanyl]acetyl}amino)-3-[({[6-methyl-2-(methylsu lfanyl)-4-pyrimidinyl]sulfanyl}methyl)sulfanyl]-8-oxo-5-thia-1-azabicyclo [4.2.0]oct-2-ene-2-carboxylic acid

The title compound(yield through two steps: 3.1%) was obtained according to the same procedure as Example 1.

¹H NMR(DMSO d₆) δ 9.21(1H, d, 8.7 Hz), 7.50˜7.46(2H, m), 7.18(1H, m), 5.50˜5.45(1H, m), 4.95(1H, d, 4.6 Hz), 4.61(1H, d, 13.3 Hz), 4.53(1H, d, 13.3 Hz), 4.01˜3.89(2H, m), 3.69(1H, d, 16.5 Hz), 3.47(1H, d, 16.9 Hz), 3.3(3H, s), 2.33(3H, s)

Mass(m/e) 634

### EXAMPLE 23

Synthsis of (6R,7R)-i,4,6-triamino-2-[({[(2-carboxy-7-({2-[(2,5-dichlo-rophenyl)sulfanyl]acetyl}a mino)-8-oxo-5-thia-1-azabicyclo [4.2.0]oct-2-en-3-yl)]sulfan-yl}methyl)sulfinyl]-5-meth ylpyrimidin-1-ium

The title compound(yield through two steps: 7.5%) was obtained according to the same procedure as Example 1.

¹H NMR(DMSO d₆) d 9.26(1H, d, 6.4 Hz), 7.95(1H, brs), 7.49˜7.46(2H, m), 7.25˜7.23(1H, m), 6.11(1H, brs), 5.47(1H, m), 4.85(1H, d, 4.6 Hz), 4.38(2H, m), 3.92(2H, m), 3.68(1H, d, 16.9 Hz), 3.46(1H, d, 16.9 Hz), 1.84(3H, s)

Mass(m/e) 634

### PREPARATION 7

Synthesis of benzhydryl (6R,7R)-3-(acetylsulfanyl)-7-({2-[(2,6-dichlo-ro-4-pyridinyl)sulfanyl]acetyl}amino)-8-oxo-5-thia-1-azabicyclo[4.2.0]oct-2-ene-2-carboxylate

6.0 g(12.66 mmol) of benzhydryl (6R,7R)-3-(acetylsulfanyl)-7-amino-8-oxo-5-thia-1-azabicyclo[4.2.0]oct-2-ene-2-carboxyl ate hydrochloric acid salt and 3.0 g(12.66 mmol) of 2-[(2,6-dichloro-4-pyridinyl)sulfanyl]acetic acid were dissolved in 50 ml of dichloromethane. After the temperature of the reaction vessel was lowered to −30° C., 2.66 ml(31.65 mmol) of pyridine and 1.56 ml(16.46 mmol) of phosphoryloxy chloride were successively added dropwise. The temperature of the reaction vessel was increased to 0° C. with stirring for 4 hours. The mixture was diluted with an excess of ethylacetate and washed with saturated ammonium chloride solution, 5% sodium bicarbonate solution and brine one by one, dried over anhydrous magnesium sulfate, and filtered. After the filtrate was evaporated under reduced pressure, the resulting residue was purified by column chromatography to give 7.0 g(yield: 85.9%) of the title compound.

¹H NMR(DMSO) δ 9.45˜9.43(1H, d, J=8.25 Hz), 8.73(1H, br, s), 7.52(2H, s), 7.50˜7.25(10H, m), 6.95(1H, s), 5.86˜5.84(1H, dd, J=5.04, 8.25 Hz), 5.27˜5.26(1H, d, J=5.04 Hz), 4.01(2H, s), 3.86˜3.83(1H, Abq, J=17.87 Hz), 3.52˜3.48(1H, Abq, J=17.87 Hz), 2.15(3H, s)

Mass(m/e) 659

### PREPARATION 8

Synthesis of benzhydryl (6R,7R)-3-[(chloromethyl)sulfanyl]-7-({2-[(2,6-dichloro-4-pyridinyl)sulfanyl]acetyl}a mino)-8-oxo-5-thia-1-azabicyclo[4.2.0]oct-2-ene-2-carboxylate

7.44 g(11.57 mmol) of benzhydryl (6R,7R)-3-(acetylsulfanyl)-7-({2-[(2,6-dichloro-4-pyridinyl)sulfanyl]acetyl}amino)-8-oxo-5-thia-1-azabicyclo[4.2.0]oct-2-ene-2-carboxylate was dissolved in 30 ml of dimethylformamide. After the temperature of the reaction vessel was lowered to −20° C., to the mixture was slowly added 1.6 ml(18.51 mmol) of morpholine. The resulting mixture was stirred at −20° C. for 1 hour. Subsequently, the mixture was diluted with ethylacetate, washed with 1% hydrochloric acid solution and brine, dried over anhydrous magnesium sulfate, and filtered. After the filtrate was evaporated under reduced pressure, the resulting residue was dissolved in 310 ml of dimethylformamide. After the temperature of the reaction vessel was lowered to −20° C., 1.71 ml(23.14 mmol) of chloroiodomethane and 1.4 ml(8.09 mmol) of diisopropylethylamine were slowly added to the mixture. The mixture was stirred at 20° C. for 24 hours. The mixture was diluted with ethylacetate, washed with 1% hydrochloric acid solution and brine, dried over anhydrous magnesium sulfate, and filtered. After the filtrate was evaporated under reduced pressure, the resulting residue was purified by column chromatography to give 5.0 g(yield: 65.0%) of the title compound.

¹H NMR(CDCl₃) δ 7.53˜7.30(10H, m), 7.08(2H, s), 6.95(1H, s), 5.76˜5.74(1H, m), 5.01(1H, d, J=4.58 Hz), 4.74˜4.72(1H, d, J=12.83 Hz), 4.60(1H, d, J=12.37 Hz), 3.87˜3.6(4H, m)

Mass(m/e) 665

### EXAMPLE 24

Synthesis of (6R,7R)-3-({[(2,6-diamino-4-pyrimidinyl)sulfanyl]meth-yl}sulfanyl)-7-({2-[(2,6-dichlo ro-4-pyridinyl)sulfanyl]acetyl}amino)-8-oxo-5-thia-1-azabicyclo[4.2.0]oct-2-ene-2-car boxylic acid

0.4 g(0.6 mmol) of benzhydryl (6R,7R)-3-[(chloromethyl)sulfanyl]-7-({2-[(2,6-dichloro-4-pyridinyl)sulfanyl]acetyl}amin o)-⁸-oxo-5-thia-1-azabicyclo[4.2.0]oct-2-ene-2-carboxylate was dissolved in 4 ml of acetone, and then 0.18 g(1.2 mmol) of sodium iodide was added thereto. The mixture was stirred at room temperature for 1 hour. After removal of the solvent under reduced pressure, the resulting residue was dissolved in ethylacetate and washed with water and brine. Subsequently, the organic layer was dried over anhydrous magnesium sulfate, and filtered. After the resulting filtrate was evaporated under reduced pressure, the remaining residue was dissolved in dimethylformamide and then 0.13 g (0.66 mmol) of 2,4-diamino-6-mercaptopyrimidine ½ sulfuric acid salt was added thereto. The resulting mixture was stirred at room temperature for 24 hours. The mixture was diluted with an excess of ethylacetate, and water was added to the diluted solution to obtain a solid. The obtained solid was filtered and collected. The filtrate was washed with water and brine, dried over anhydrous magnesium sulfate, and filtered. After the filtrate was evaporated under reduced pressure, the resulting residue was dissolved in a small amount of methylene chloride, purified with diethyl ether, and filtered. The filtered solid was collected. Each solid collected above was dried under nitrogen atmosphere.

0.3 g of the solid thus obtained was deprotected with trifluoroacetic acid, anisole and triethylsilane, and then separated and purified by high pressure fractional liquid chromatography to give 23 g(yield through two steps: 6.0%) of the title compound.

¹H NMR(DMSO, 500 MHz) δ 9.26(1H, d, J=7.8 Hz, NH), 7.52(2H, s), 6.28(2H, brs, NH₂), 5.95(2H, brs, NH₂), 5.86(1H, s), 4.92(1H, d, J=4.6 Hz), 4.28˜4.39(2H, m), 3.99˜4.01(2H, m), 3.68(1H, d, J=16.9 Hz), 3.47(1H, d, J=16.9 Hz)

Mass(m/e) 605

### EXAMPLE 25

Synthesis of (6R,7R)-3-({[(4,6-diamino-2-pyrimidinyl)sulfanyl]meth-yl}sulfanyl)-7-({2-[(2,6-dichlo ro-4-pyridinyl)sulfanyl]acetyl}amino)-8-oxo-5-thia-1-azabicyclo [4.2.0]oct-2-ene-2-car boxylic acid

After 0.3 g(0.45 mmol) of benzhydryl (6R,7R)-3-[(chloromethyl)sulfanyl]-7-({2-[(2,6-dichloro-4-pyridinyl)sulfanyl]acetyl}amin o)-8-oxo-5-thia-1-azabicyclo[4.2.0]oct-2-ene-2-carboxylate was dissolved in 3ml of dimethylformamide, 0.13 g(0.9 mmol) of sodium iodide was added thereto and 0.08 g(0.58 mmol) of 4,6-diamino-2-pyrimidinethiole was added to the reaction mixture. The mixture was stirred at room temperature for 24 hours. The mixture was diluted with an excess of ethylacetate, and water was added to the diluted solution to obtain a solid. The obtained solid was filtered and collected. The filtrate was washed with water and brine, dried over anhydrous magnesium sulfate, and filtered. After the filtrate was evaporated under reduced pressure, the resulting residue was dissolved in a small amount of methylene chloride, purified with diethyl ether, and filtered. The filtered solid was collected. Each solid collected above was dried under nitrogen atmosphere.

0.17 g of the solid thus obtained was deprotected with trifluoroacetic acid, anisole and triethylsilane, and then separated and purified by high pressure fractional liquid chromatography to give 23 g(yield through two steps: 8.4%) of the title compound.

¹H NMR(D₂O, 400 MHz) δ 7.02(2H, s), 5.26(1H, s), 5.15(1H, s), 4.73(1H, s), 4.15(2H, s), 3.45˜3.49, 3.25˜3.29(2H, Abq, J=16.4 Hz)

Mass(m/e) 605

### EXAMPLE 26

Synthesis of (6R,7R)-3-({[(4-amino-1H-pyrazolo[3,4-d]pyrimidin-6-yl)sulfanyl]methyl}sulfanyl)-7-({2-[(2,6-dichloro-4-pyridinyl)sulfanyl]acetyl}amino)-8-oxo-5-thia-1-azabicyclo[4.2.0]oct-2-ene-2-carboxylic acid

The title compound(yield through two steps: 12.2%) was obtained according to the same procedure as Example 25.

¹H NMR(D₂O, 400 MHz) δ 7.93(1H, s), 7.25(2H, s), 5.78(1H, m), 5.10(1H, m), 4.25˜4.34(2H, m), 3.82˜3.87(1H, m), 3.57˜3.71(2H, m)

Mass(m/e) 630

### EXAMPLE 27

Synthesis of (6R,7R)-7-({2-[(2,6-dichloro-4-pyridinyl)sulfanyl]acetyl}a-mino)-8-oxo-3-{[(1H-pyraz olo[3,4-d]pyrimidin-4-yl-sulfanyl)methyl]sulfanyl}-5-thia-1-azabicyclo[4.2.0]oct-2-ene -2-carboxylic acid

The title compound(yield through two steps: 18.0%) was obtained according to the same procedure as Example 25.

¹H NMR(DMSO, 500 MHz) δ 9.24(1H, d, J=8.3 Hz, NH), 8.70(1H, s), 8.24(1H, s), 7.52(2H, s), 5.44˜5.46(1H, m), 4.95(1H, d, J=5.0 Hz), 4.83˜4.85, 4.71˜4.74(2H, Abq, J=13.1 Hz), 3.96˜4.03(2H, m), 3.70˜3.74, 3.49˜3.52(2H, Abq, J=16.5 Hz)

Mass(m/e) 615

### EXAMPLE 28

Synthesis of (6R,7R)-3-({[(2-amino-6-hydroxy-4-pyrimidinyl)sulfanyl]m-ethyl}sulfanyl)-7-({2-[(2,6-dichloro-4-pyridinyl)sulfanyl]acetyl}amino)-8-oxo-5-thia-1-azabicyclo[4.2.0]oct-2-en e-2-carboxylic acid

The title compound(yield through two steps: 11.7%) was obtained according to the same procedure as Example 25.

¹H NMR(D₂O, 400 MHz) δ 7.05(2H, s), 5.51(1H, s), 5.24(1H, d, J=4.4 Hz), 4.75(1H, d, J=4.4 Hz), 4.00˜4.10(2H, m), 3.67˜3.72, 3.47˜3.52(2H, Abq, J=20 Hz), 3.43˜3.48, 3.18˜3.22(2H, Abq, J=17.2 Hz)

Mass(m/e) 606

### EXAMPLE 29

Synthesis of (6R,7R)-7-({2-[(2,6-dichloro-4-pyridinyl)sulfanyl]acetyl}a-mino)-3-[({[2-(ethylsulfanyl )-6-methyl-4-pyrimidinyl]sulfanyl)methyl]sulfanyl}-8-oxo-5-thia-1-azabicyclo [4.2.0]oc t-2-ene-2-carboxylic acid

The title compound(yield through two steps: 14.4%) was obtained according to the same procedure as Example 25.

¹H NMR(DMSO, 400 MHz) δ 9.25(1H, d, J=8.0 Hz, NH), 7.52(2H, s), 7.17(1H, s), 5.46˜5.49(1H, m), 4.95(1H, d, J=4.0 Hz), 4.60˜4.63, 4.50˜4.54(2H, Abq, J=12.0 Hz), 3.96˜4.05(2H, Abq, J=15.6 Hz), 3.67˜3.72, 3.44˜3.49(2H, Abq, J=16.8 Hz), 3.09(2H, q, J=8.0 Hz), 2.33(3H, s), 1.31(3H, t, J=8.0 Hz)

Mass(m/e) 649

### EXAMPLE 30

Synthesis of 7-amino-5-[({[(6R,7R)-2-carboxy-7-({2-[(2,6-dichloro-4-pyridinyl)sulfanyl]acetyl}ami no)-8-oxo-5-thia-i-azabicyclo[4.2.0]oct-2-en-3-yl]sulfan-yl}methyl)sulfanyl]-1H-[1,2,4]triazolo[1,5-c]pyrimidin-4-ium

The title compound(yield through two steps: 15.5%) was obtained according to the same procedure as Example 25.

¹H NMR(D₂O, 400 MHz) δ 8.01(1H, s), 7.10(2H, s), 6.17(1H, s), 5.39(1H, d, J=4.4 Hz), 4.91(1H, d, J=4.4 Hz), 4.56(2H, m), 3.65˜3.70, 3.41˜3.45(2H, Abq, J=17.0 Hz)

Mass(m/e) 631

### EXAMPLE 31

Synthesis of 2,7-diamino-5-[({[(6R,7R)-2-carboxy-7-({2-[(2,6-dichloro-4-pyridinyl)sulfanyl]acetyl}amino)-8-oxo-5-thia-1-azabicyclo [4.2.0]oct-2-en-3-yl]sulfan-yl}methyl)sulfanyl]-1-met hyl-1H-[1,2,4]triazolo[1,5-c]pyrimidin-4-ium

The title compound(yield through two steps: 0.13%) was obtained according to the same procedure as Example 25.

¹H NMR(DMSO, 400 MHz) δ 9.26(1H, d, J=8.0 Hz, NH), 7.69(2H, brs), 7.52(2H, s), 6.17(1H, s), 5.45-5.48(1H, m), 4.87(1H, d, J=4.8 Hz), 4.62˜4.72(2H, m), 4.00(2H, s), 3.55-3.71(2H, m),

Mass(m/e) 660

### EXAMPLE 32

Synthesis of (6R,7R)-7-({2-[(2,6-dichloro-4-pyridinyl)sulfanyl]acetyl}am-ino)-3-{[({(4-hydroxy-6-[(2-hydroxyethyl)amino]-2-pyrimidinyl}sulfanyl)methyl]sulfa-nyl}-8-oxo-5-thia-1-azabi cyclo[4.2.0]oct-2-ene-2-carboxylic acid

The title compound(yield through two steps: 15.9%) was obtained according to the same procedure as Example 25.

¹H NMR(D₂O, 400 MHz) δ 7.28(2H, s), 5.51(1H, d, J=4.4 Hz), 5.08(1H, s), 4.99(1H, d, J=4.8 Hz), 4.48(2H, s), 3.92˜4.02 (2H, m), 3.72˜3.76, 3.46˜3.51(2H, Abq, J=1 7.4), 3.68(2H, t), 3.35(2H, bs)

Mass(m/e) 650

### EXAMPLE 33

Synthesis of 2,4-diamino-6-[({[(6R,7R)-2-carboxy-7-({2-[(2,6-dichloro-4-pyridinyl)sulfanyl]acetyl}amino)-8-oxo-5-thia-1-azabicyclo[4.2.0]oct-2-en-3-yl]sulfan-yl}methyl)sulfanyl]-1-met hylpyrimidin-1-ium

After 0.38 g(0.575 mmol) of benzhydryl (6R,7R)-3-[(chloromethyl)sulfanyl]-7-({2-[(2,6-dichloro-4-pyridinyl)sulfanyl]acetyl}amin o)-8-oxo-5-thia-1-azabicyclo[4.2.0]oct-2-ene-2-carboxylate was dissolved in 5 ml of dimethylformamide, 0.17 g(1.149 mmol) of sodium iodide was added thereto and 0.11 g(0.632 mmol) of 2,6-diamino-3-methyl-4(3H)-pyrimidinethione was added to the reaction mixture. The mixture was stirred at room temperature for 24 hours. The mixture was diluted with an excess of ethylacetate. The diluted solution was washed with water three times, dried over anhydrous magnesium sulfate, and filtered. After the filtrate was evaporated under reduced pressure, the resulting residue was purified with diethyl ether and then dried under nitrogen atmosphere to a solid.

0.15 g of the solid thus obtained was deprotected with trifluoroacetic acid, anisole and triethylsilane, and then separated and purified by high pressure fractional liquid chromatography to give 0.014 g(yield through two steps: 4.1%) of the title compound.

¹HNMR(DMSO-d₆) δ 9.23˜9.21(1H, d, J=8.25 Hz), 7.89(1H, br, s), 7.52(2H, s), 7.32˜7.35(1H, d, J=15.58 Hz), 5.51(1H, s), 5.46˜5.45(1H, d, J=5.04 Hz), 4.97˜4.96(1H, d, J=5.04 Hz), 4.00˜3.96(3H, m), 3.85˜3.84(1H, m), 3.40˜3.34(4H, m), 1.22(3H, t)

Mass(m/e) 620

### EXPERIMENT 1

Minimum Inhibitory Concentration (MIC)

The effectiveness of the compound according to the present invention was determined by obtaining Minimum Inhibitory Concentration (MIC) of the compounds prepared by the above Examples (I-1 to I-33) and vancomycin, which is the known compound having a potent activity against gram-positive strains, as the control drug against the standard strains. Specifically, Minimum Inhibitory Concentration was obtained by diluting the test material with a double dilution method, dispersing them in Mueller-Hinton agar medium, inoculating each of the test strain having 10⁷ cfu (colny forming unit) in an amount of 2:1 to the medium and then incubating them at 37° C. for 20 hours. The results are shown in the following Tables 1 and 2.

TABLE 1
Sensitivity test result using standard strains (:g/ml)

| | | S. | S. | | |
|---|---|---|---|---|---|
| | Staphylococcus | aureus | aureus | S. epidermidis | E. faecalis |
| | aureus giorgio | 77 | 241 | R005 | L239 |
| 1-1 | <0.008 | 0.063 | 1 | 0.063 | 0.25 |
| 1-2 | <0.008 | 0.13 | 1 | 0.063 | 0.25 |
| I-3 | <0.008 | 0.25 | 8 | 0.13 | 0.5 |
| I-4 | <0.008 | 0.25 | 4 | 0.25 | 0.5 |
| I-5 | 0.016 | 0.5 | 8 | 0.5 | 1 |
| I-6 | 0.031 | 0.5 | 8 | 0.5 | 4 |
| I-7 | <0.008 | 0.13 | 2 | 0.063 | 0.13 |
| I-8 | 0.016 | 0.25 | 8 | 0.25 | 0.25 |
| I-9 | <0.008 | 0.25 | 4 | 0.25 | 0.25 |
| I-10 | <0.008 | 0.25 | 4 | 0.13 | 0.5 |
| I-11 | <0.008 | 0.063 | 1 | 0.063 | 0.25 |
| I-12 | 0.031 | 0.5 | 4 | 0.5 | 0.25 |
| I-13 | <0.008 | 0.13 | 2 | 0.13 | 0.25 |
| I-14 | <0.008 | 0.25 | 2 | 0.13 | 0.13 |
| I-15 | <0.008 | 0.13 | 1 | 0.13 | 0.25 |
| I-16 | <0.008 | 0.25 | 2 | 0.063 | 0.5 |
| I-17 | 0.016 | 0.13 | 2 | 0.25 | 0.5 |
| I-18 | 0.016 | 0.25 | 4 | 0.25 | 0.5 |
| I-19 | 0.063 | 4 | 32 | 2 | 4 |
| I-20 | <0.008 | 1 | 8 | 0.5 | 0.5 |
| I-21 | 0.016 | 1 | 8 | 0.5 | 0.5 |
| I-22 | <0.008 | 0.5 | 4 | 0.25 | 0.25 |
| I-23 | 0.5 | 8 | >32 | 8 | 16 |
| Vanco- | 1 | 1 | 2 | 1 | 2 |
| mycin | | | | | |

TABLE 2
Sensitivity test result using standard strains (:g/ml)

| | S. aureus | S. aureus | S. aureus | S. epidermidis | E. faecalis |
|---|---|---|---|---|---|
| | giorgio | 77 | K311 | R005 | EFS004 |
| 1-24 | 0.016 | 0.25 | 0.5 | 0.25 | 0.5 |
| 1-25 | 0.016 | 0.25 | 0.5 | 0.25 | 0.5 |
| I-26 | 0.016 | 0.25 | 0.5 | 0.25 | 0.5 |
| I-27 | 0.13 | 0.25 | 0.5 | 0.25 | 0.5 |
| I-28 | 0.031 | 0.25 | 0.5 | 0.25 | 1 |
| I-29 | 0.031 | 0.5 | 2 | 1 | 0.25 |
| I-30 | 0.031 | 0.5 | 1 | 0.5 | 0.5 |
| I-31 | 0.031 | 0.25 | 0.5 | 0.25 | 0.5 |
| I-32 | 0.13 | 0.1 | 1 | 1 | 1 |
| I-33 | 0.031 | 0.25 | 1 | 0.25 | 0.5 |
| Vanco- | 1 | 1 | 2 | 1 | 2 |
| mycin | | | | | |

From the result of Minimum Inhibitory Concentration test, it can be seen that the compound according to the present invention has a good activity against major pathogenic microorganisms, which cause hospital infection, including MRSA strains.

While the invention has been described with respect to the above specific embodiments, it should be recognized that various modifications and changes can be made to the invention by those skilled in the art, which also fall within the scope of the invention as defined by the appended claims.

What is claimed is:

1. A cephalosporin compound represented by the following formula (I): 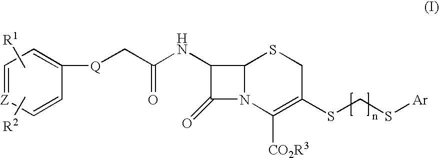
in which
R¹ and R² each independently represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;
R³ represents hydrogen or a carboxy-protecting group;
Q represents S, O, CH₂, NH or NR wherein R is hydrogen, C₁₋₆ alkyl or benzyl;
Z represents CH or N;
n denotes an integer of 1 or 2; and
Ar represents a heteroaryl group represented by one of the following formulas: 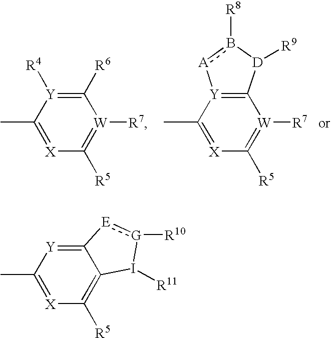
wherein X, Y, W, A, B, D, E, G and I independently of one another represent N or C (or CH), provided that the six-membered ring forms a pyrimidine structure;
R⁴ represents hydrogen, C₁₋₄ alkyl, or amino substituted or unsubstituted with a substitutent selected from the group consisting of C₁₋₆ alkyl and C₁₋₆ hydroxyalkyl;
R⁵ and R⁶ each independently represent hydrogen, hydroxy, C₁₋₄ alkyl, C₁₋₆ alkylthio substituted or unsubstituted with a substitutent selected from the group consisting of C₁₋₆ alkyl, C₁₋₆ hydroxyalkyl and C₁₋₆ aminoalkyl, or amino substituted or unsubstituted with a substitutent selected from the group consisting of C₁₋₆ alkyl, C₁₋₆ hydroxyalkyl and C₁₋₆ aminoalkyl;
R⁷, R⁸, R⁹, R¹⁰ and R¹¹ independently of one another represent hydrogen, C₁₋₆ alkyl, or amino substituted or unsubstituted with a substitutent selected from the group consisting of C₁₋₆ alkyl, C₁₋₆ hydroxyalkyl and C₁₋₆ aminoalkyl; and [symbol]
denotes a single bond or a double bond;
or pharmaceutically acceptable non-toxic salt, physiologically hydrolysable ester, hydrate, solvate or isomer thereof.

2. The compound of claim 1, wherein the compound is selected from the group consisting of
(6R,7R)-3-({[(2,6-diamino-4-pyrimidinyl)sulfanyl]methyl}sulfanyl)-7-({2-[(2, 5-dichlorophenyl)sulfanyl]acetyl}amino)-8-oxo-5-thia-1-azabicyclo[4.2.0]oct-2-ene-2-carboxylic acid,
(6R,7R)-3-({[(2-amino-6-hydroxy-4-pyrimidinyl)sulfanyl]methyl}sulfanyl)-7-({2-[(2,5-dic hlorophenyl)sulfanyl]acetyl}amino)-8-oxo-5-thia-1-azabicyclo[4.2.0]oct-2-e-ne-2-carboxy lic acid,
(6R,7R)-3-({[(2-amino-6-hydroxy-4-pyrimidinyl)sulfanyl]methyl}sulfanyl)-7-({2-[(2,6-dic hlorophenyl)sulfanyl]acetyl}amino)-8-oxo-5-thia-1-azabicyclo[4.2.0]oct-2-e-ne-2-carboxy lic acid,
(6R,7R)-3-({[(6-amino-2-hydroxy-4-pyrimidinyl)sulfanyl]methyl}sulfanyl)-7-({2-[(2,5-dic hlorophenyl)sulfanyl]acetyl}amino)-8-oxo-5-thia-1-azabicyclo [4.2.0]oct-2-e-ne-2-carboxy lic acid,
(6R,7R)-3-({[(2,6-diamino-4-pyrimidinyl)sulfanyl]methyl}sulfanyl)-7- {[2-[(2,5-dichloroa nilino)acetyl]amino}-8-oxo-5-thia-1-azabicyclo[4.2.0]oct-2-ene-2-carboxylic acid,
(6R,7R)-3-({[(2,6-diamino-4-pyrimidinyl)sulfanyl]methyl}sulfanyl)-7-({2-[2, 5-dichloro(methyl)anilino]acetyl}amino)-8-oxo-5-thia-1-azabicyclo[4.2.0]oct-2-ene-2-carboxylic acid,
1,4-diamino-2-[({[(6R,7R)-2-carboxy-7-({2-[(2,5-dichlorophenyl)sulfanyl]ac-etyl}amino)-8-oxo-5-thia-1-azabicyclo [4.2.0]oct-2-en-3-yl)sulfanyl}methyl)sulfanyl]pyri-midin-1-ium,
(6R,7R)-7-amino-5-[({[2-carboxy-7-({2-[(2,5-dichlorophenyl)sulfanyl]acetyl}amino)-8-oxo-5-thia-1-azabicyclo [4.2.0]oct-2-en-3-yl]sulfanyl}methyl)sulfanyl]-3H-[1,2, 4]triazolo[1,5-c]pyrimidin-4-ium,
(6R,7R)-3-({[(4-amino-6,7-dihydro-5H-cyclopenta[d]pyrimidin-2-yl)sulfanyl]methyl}sulfanyl)-7-({2-[(2,5-dichlorophenyl)sulfanyl]acetyl}amino)-8-oxo-5-thia-1-aza-bi cyclo[4.2.0]oct-2-ene-2-carboxylic acid,
(6R,7R)-1,4,6-triamino-2-[({[2-carboxy-7-({2-[(2,5-dichlorophenyl)sulfanyl]acetyl}amino)-8-oxo-5-thia-1-azabicyclo[4.2.0]oct-2-en-3-yl]sulfanyl}methyl)sulfanyl]p-y rimidin-1-ium,
(6R,7R)-1,2-diamino-4-[({(E)-3-[2-carboxy-7-({2-[(2,5-dichlorophenyl)sulf-anyl]acetyl}a mino)-8-oxo-5-thia-1-azabicyclo [4.2.0]oct-2-en-3-yl]sulfanyl}methyl)sulfan-yl]-6,7-dihyd ro-5H-cyclopenta[d]pyrimidin-1-ium,
(6R,7R)-7-({2-[(2,5-dichlorophenyl)sulfanyl]acetyl}amino)-3-[({[2-(ethylsul-fanyl)-6-met hyl-4-pyrimidinyl]sulfanyl}methyl)sulfanyl]-8-oxo-5-thia-1-azabicyclo[4.2. 0]oct-2-ene-2-carboxylic acid,
(6R,7R)-3-({[(7-amino-1H-pyrazolo[4,3-d]pyrimidin-5-yl)sulfanyl]methy-1}sulfanyl)-7-({2-[(2,5-dichlorophenyl)sulfanyl]acetyl}amino)-8-oxo-5-thia-1-azabicyc-lo [4.2.0]oct-2-ene-2-carboxylic acid,
(6R,7R)-2,7-diamino-5-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-methyl-1H,2H, 3H-[1,2,4]triazolo[1,5-c]pyrimidin-4-ium,
(6R,7R)-2,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-methylpyrimidi n-1-ium,
(6R,7R)-3-({[(4,6-diamino-2-pyrimidinyl)sulfanyl]methyl}sulfanyl)-7-({2-[(2,5-dichlorop henyl)sulfanyl]acetyl}amino)-8-oxo-5-thia-1-azabicyclo[4.2.0]oct-2-ene-2-carboxylic acid,
(6R,7R)-3-({[(5,6-diamino-4-pyrimidinyl)sulfanyl]methyl}sulfanyl)-7-({2-[(2,5-dichlorop henyl)sulfanyl]acetyl}amino)-8-oxo-5-thia-1-azabicyclo[4.2.0]oct-2-ene-2-carboxylic acid,
(6R,7R)-3-({[(4,6-diamino-5-methyl-2-pyrimidinyl)sulfanyl]methyl}sulfan-yl)-7-({2-[(2,5-dichlorophenyl)sulfanyl]acetyl}amino)-8-oxo-5-thia-1-azabicyclo[4.2.0]oc-t-2-ene-2-carb oxylic acid,
(6R,7R)-1,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-3-methyl-2-(meth ylamino)-1,3,5-triazine-1,3-diium,
(6R,7R)-2,4-diamino-6-[({[2-carboxy-7-({2-[(2,5-dichlorophenyl)sulfany-1]acetyl}amino)-8-oxo-5-thia-1-azabicyclo[4.2.0]oct-2-en-3-yl]sulfanyl}methyl)sulfanyl]-1-ethyl-1,3,5-tria zinc 1,3-diium,
(6R,7R)-5-[({[(2-carboxy-7-({2-[(2,5-dichlorophenyl)sulfanyl]acetyl}amino)-8-oxo-5-thia-1azabicyclo[4.2.0]oct-2-en-3-yl)]sulfanyl}methyl)sulfinyl]-1H-[1,2,4]triazo-lo[3,4-b][1,3 ,5]triazine-4-ium,
(6R,7R)-7-({2-[(2,5-dichlorophenyl)sulfanyl]acetyl}amino)-3-[({[6-methyl-2-(methylsulfa nyl)-4-pyrimidinyl]sulfanyl}methyl)sulfanyl]-8-oxo-5-thia-1-azabicyclo [4.2. 0]oct-2-ene-2-carboxylic acid,
(6R,7R)-1,4,6-triamino-2-[({[(2-carboxy-7-({2-[(2,5-dichlorophenyl)sulfan-yl]acetyl}amin o)-8-oxo-5-thia-1-azabicyclo[4.2.0]oct-2-en-3-yl)]sulfanyl}methyl)sulfin-yl]-5-methylpyri midin-1-ium,
(6R,7R)-3-({[(2,6-diamino-4-pyrimidinyl)sulfanyl]methyl}sulfanyl)-7-({2-[(2,6-dichloro-4-pyridinyl)sulfanyl]acetyl}amino)-8-oxo-5-thia-1-azabicyclo [4.2.0]oct-2-ene-2-carboxylic acid,
(6R,7R)-3-({[(4,6-diamino-2-pyrimidinyl)sulfanyl]methyl}sulfanyl)-7-({2-[(2,6-dichloro-4-pyridinyl)sulfanyl]acetyl}amino)-8-oxo-5-thia-1-azabicyclo[4.2.0]oct-2-ene-2-carboxylic acid,
(6R,7R)-3-({[(4-amino-i H-pyrazolo[3,4-d]pyrimidin-6-yl)sulfanyl]methy-1}sulfanyl)-7-({2-[(2,6-dichloro-4-pyridinyl)sulfanyl]acetyl}amino)-8-oxo-5-thia-1-azabi-cyclo[4.2.0]oct-2-ene-2-carboxylic acid,
(6R,7R)-7-({2-[(2,6-dichloro-4-pyridinyl)sulfanyl]acetyl}amino)-8-oxo-3- {[(1H-pyrazolo[3 ,4-d]pyrimidin-4-yl-sulfanyl)methyl]sulfanyl}-5-thia-1-azabicyclo[4.2. 0]oct-2-ene-2-carboxylic acid,
(6R,7R)-3-({[(2-amino-6-hydroxy-4-pyrimidinyl)sulfanyl]methyl}sulfanyl)-7-({2-[(2,6-dic hloro-4-pyridinyl)sulfanyl]acetyl}amino)-8-oxo-5-thia-1-azabicyclo[4.2.0]-oct-2-ene-2-car [symbol]
denotes a single bond or a double bond;
(6R,7R)-7-({2-[(2,6-dichloro-4-pyridinyl)sulfanyl]acetyl}amino)-3-[({[2-(ethylsulfanyl)-6-methyl-4-pyrimidinyl]sulfanyl)methyl]sulfanyl}-8-oxo-5-thia-1-azabicy-clo[4.2.0]oct-2-en e-2-carboxylic acid,
7-amino-5-[({[(6R,7R)-2-carboxy-7-({2-[(2,6-dichloro-4-pyridinyl)sulfany-1]acetyl}amino )-8-oxo-5-thia-1-azabicyclo[4.2.0]oct-2-en-3-yl]sulfanyl}methyl)sulfanyl]-1H-[1,2,4]triazo lo[1,5-c]pyrimidin-4-ium,
2,7-diamino-5-[({[(6R,7R)-2-carboxy-7-({2-[(2,6-dichloro-4-pyridinyl)sulf-anyl]acetyl}a mino)-8-oxo-5-thia-1-azabicyclo[4.2.0]oct-2-en-3-yl]sulfanyl}methyl)sulfan-yl]-1-methyl-1H-[1,2,4]triazolo[1,5-c]pyrimidin-4-ium,
(6R,7R)-7-({2-[(2,6-dichloro-4-pyridinyl)sulfanyl]acetyl}amino)-3- {[({4-hy-droxy-6-[(2-h ydroxyethyl)amino]-2-pyrimidinyl}sulfanyl)methyl]sulfanyl}-8-oxo-5-thia-1-azabicyclo [4. 2.0]oct-2-ene-2-carboxylic acid, and
2,4-diamino-6-[({[(6R,7R)-2-carboxy-7-({2-[(2,6-dichloro-4-pyridinyl)sulf-anyl]acetyl}a mino)-8-oxo-5-thia-1-azabicyclo[4.2.0]oct-2-en-3-yl]sulfanyl}methyl)sulfan-yl]-1-methylp yrimidin-1-ium.

3. A process for preparing the compound of formula (I) according to claim 1, which comprises reacting a compound of formula (V): 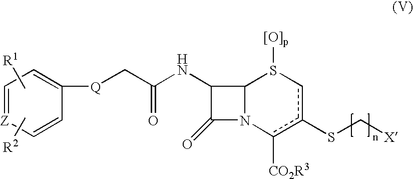
in which R¹, R², R³, Z, Q and n are as defined in claim 1, p denotes an integer of 0 or 1, and X′ represents halogen atom, with a compound of formula (VI):
HS-Ar (VI)
in which Ar is as defined in claim 1, optionally followed by addition of alkali metal to obtain a compound of formula (VII): 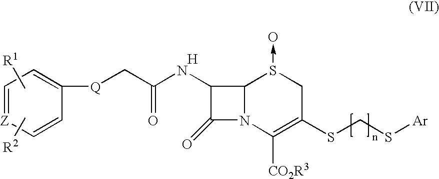
in which R¹, R², R³, Z, Q, n and Ar are as defined in claim 1, and reducing S→oxide of the compound of formula (VII).

4. The process of claim 3, which further comprises removing acid-protecting group.

5. An antibiotic composition containing the compound of formula (I) or its pharmaceutically acceptable salt according to claim 1 as an active ingredient, together with a pharmaceutically acceptable carrier.

6. The antifungal composition of claim 5, wherein the unit dosage form contains the compound of formula (I) according to claim 1 in an amount of about 50 to 1,500 mg.
